(19) United States
(12) Patent Application Publication
(10) Pub. No.: US 2005/0080076 A1
(43) Pub. Date: Apr. 14, 2005

(54) N-desmethyl levomepromazine

(75) Inventors: Keith A. Moore, Loveland, OH (US); Ralph A. Heasley, Union, KY (US)

Correspondence Address: WOOD, HERRON & EVANS, LLP, 2700 CAREW TOWER, 441 VINE STREET, CINCINNATI, OH, 45202, US

(73) Assignee: Xanodyne Pharmacal, Inc., Florence, KY (US)

(21) Appl. No.: 10/681,431

(22) Filed: Oct. 8, 2003

(51) Int. Cl. A61K 31/5415

(52) U.S. Cl. 514/226.2

(57) ABSTRACT

A pharmaceutically active N-desmethyl levomepromazine (abbreviated NDM LMP) and method of use. NDM LMP has substantially the same therapeutic effects as the parent levomepromazine but is subject to less disposition (e.g. presystemic and systemic metabolism) and thus has improved properties over the parent.

### FIELD OF THE INVENTION

The invention relates to clinical uses of N-desmethyl levomepromazine as a therapeutic agent.

### BACKGROUND

Levomepromazine (LMP), also called methotrimeprazine (MPZ), is an antagonist for various receptors. Specifically, LMP is an antagonist primarily for dopamine, serotonin, histamine, α adrenergic and muscarinic receptors. Binding of LMP to these receptors thus reduces or inhibits the effects elicited by receptor agonists.

In vivo, LMP undergoes extensive metabolism both in the liver and in the intestinal cell wall. At least five different primary metabolites of the parent LMP are formed through the processes of N-dealkylation, O-dealkylation, sulfoxidation and hydroxylation (both side chain and aromatic ring). Two of these metabolites, N-desmethyl levomepromazine (NDM LMP) and levomepromazine sulfoxide, are found to have appreciable serum concentrations after administration of LMP.

### SUMMARY OF THE INVENTION

A pharmaceutical composition comprising the R(+) enantiomer, (dextrorotatory optical isomer) of 2-methoxy-10-(2-methyl-3-monomethylaminopropyl)phenothiazine, referred to herein as N-desmethyl levomepromazine, abbreviated NDM LMP but also known as N-monodesmethyl levomepromazine, and methods of using NDM LMP to inhibit agonist-modulated functions of LMP and/or NDM LMP receptors, including dopamine, serotonin, histamine, α adrenergic and muscarinic receptors are described. Because administration of NDM LMP unexpectedly and desirably eliminates formation of the sulfoxide metabolite, which occurs when the parent levomepromazine (LMP) is administered, administration of NDM LMP provides greater dopamine and serotonin receptor antagonism, with less histamine and α₁ adrenergic receptor antagonism, compared to the parent LMP. This effect occurs regardless of the route by which NDM LMP is administered, but is particularly significant when NDM LMP is administered orally. Clinically, NDM LMP has efficacy as an analgesic, antiemetic, antipsychotic, sedative, anxiolytic, antisialogogic, amnesic, anti-pruritic, antihypertensive compound, an agent for migraine therapy, and an agent to control the symptoms of benign prostatic hyperplasia (BPH). In one embodiment, one or more of these effects may be preferentially selected based upon the dose of NDM LMP. In another embodiment, the sedation and antihypertensive effects of the sulfoxide metabolite may be minimized or reduced by administering NDM LMP. In this embodiment, administration of NDM LMP would desirably have less potential to cause drowsiness (sedation) and lowered blood pressure (BP) in a patient.

The NDM LMP and sulfoxide metabolites of LMP are pharmacologically active. The NDM LMP metabolite binds to the same receptors with a comparable affinity as the parent LMP. The sulfoxide metabolite binds only to histamine and α₁ adrenergic receptors to any significant degree. The use of NDM LMP as a therapeutic entity has not previously been reported.

### BRIEF DESCRIPTION OF THE DRAWINGS

FIG. 1 shows the chemical structure of N-desmethyl levomepromazine (NDM LMP).

FIG. 2 shows levomepromazine (LMP) metabolites and potential metabolic pathways.

FIG. 3 shows NDM LMP metabolites and potential metabolic pathways.

### DETAILED DESCRIPTION

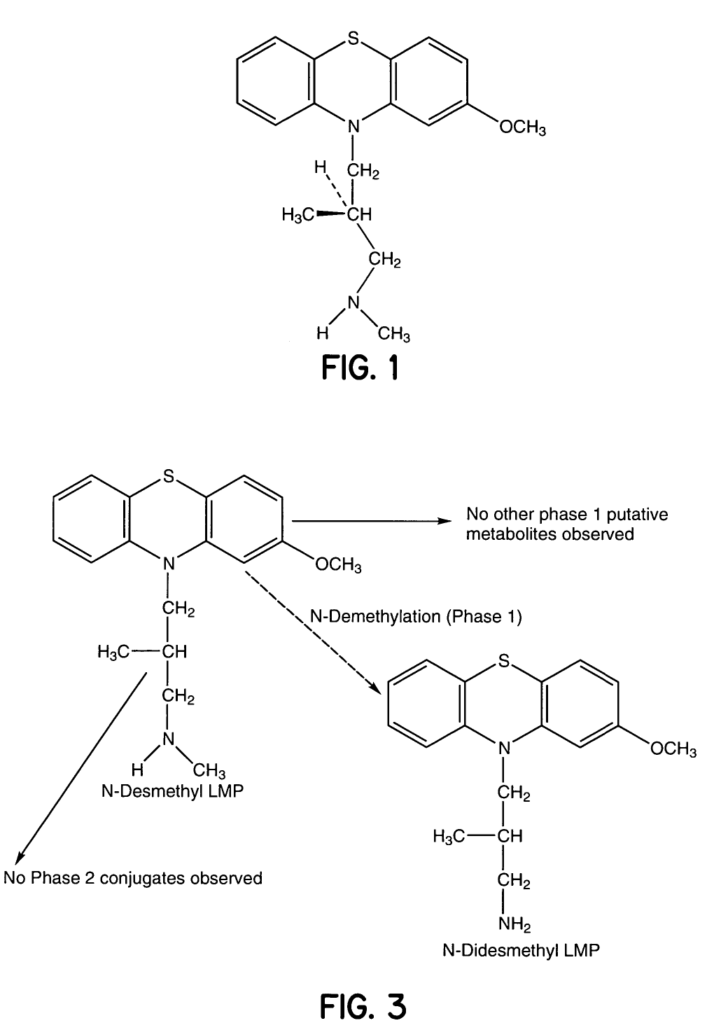

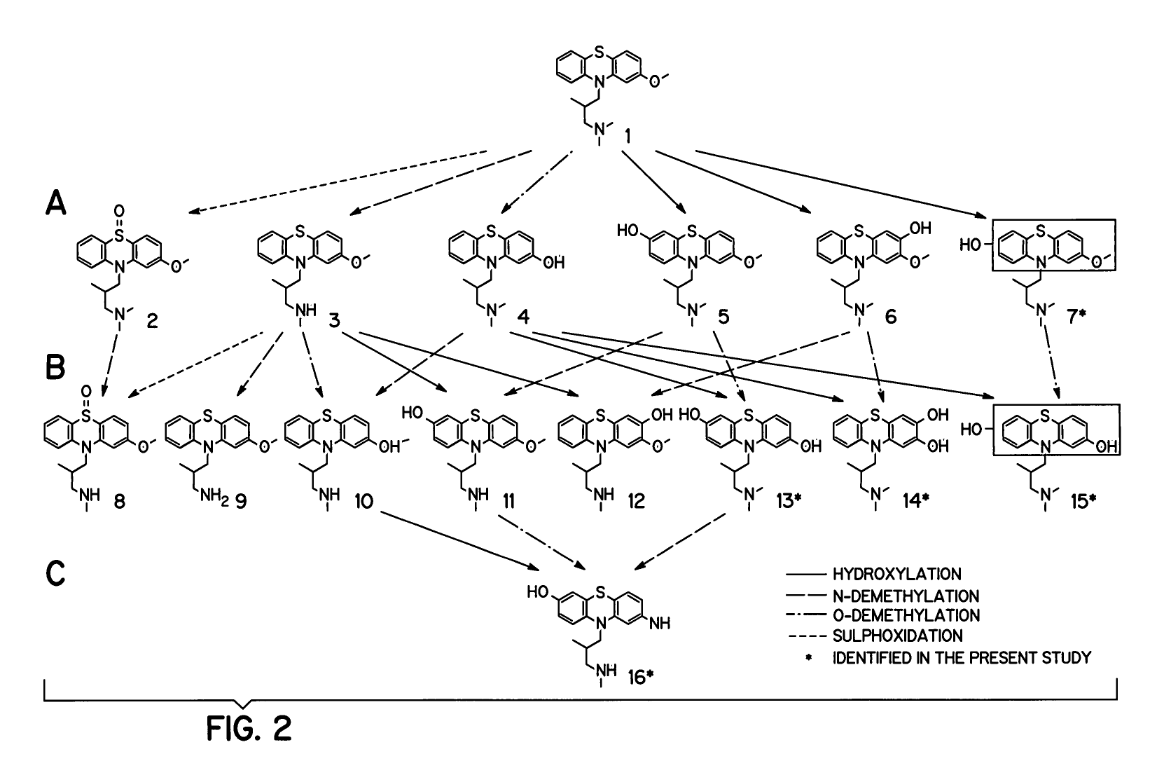

A pharmaceutically acceptable formulation of N-desmethyl levomepromazine (R(+) 2-methoxy-10-2-methyl-3-monomethylaminopropyl phenothiazine, also referred to as N-monodesmethyl levomepromazine (NDM LMP)), the chemical structure of which is shown in FIG. 1, and methods of using NDM LMP to provide pharmacologic activity, are disclosed. As used herein, NDM LMP refers to the R enantiomer, dextrorotatory optical isomer. The designation NDM LMP encompasses the free base form as well as any pharmaceutically acceptable salts. As shown in FIG. 2 (Hals and Dahl, Europ. J. Drug Metab. Pharmacokinetics 20:61 (1995)), in vivo, NDM LMP is a naturally occurring metabolite of levomepromazine (LMP). With reference to FIGS. 2, A, B, and C indicate metabolites formed by one, two and three metabolic steps, respectively. The following abbreviations are used: 1=levomepromazine (LMP); 2=LM sulfoxide; 3=N-desmethyl LMP; 4=O-desmethyl LPM; 5=7-hydroxy LMP; 6=3-hydroxy LMP; 7=‘ring-hydroxy LMP’; 8=N-desmethyl LMP sulfoxide; 9=N-didesmethyl LMP; 10=N,O-didesmethyl LMP; N=desmethyl 7-hydroxy LMP; 12=N-desmethyl 3-hydroxy LMP; 13=O-desmethyl 7-hydroxy LMP; 14=O-desmethyl 3-hydroxy LMP; 15=O-desmethyl ‘ring-hydroxy LMP’; 16=N,O-didesmethyl LMP. Conjugates of the metabolites containing hydroxyl groups are not indicated.

NDM LMP binds with substantially equivalent affinity to dopamine, serotonin, histamine, α adrenergic, and muscarinic receptors as the parent LMP, achieves comparable serum concentration as the parent LMP, exhibits comparable serum protein binding (99%) as the parent LMP, and results in at least substantially equivalent antagonist activity as the parent LMP. NDM LMP, compared to the parent is metabolically more stable to biotransformation, has improved oral absorption characteristics, reduced clearance variability, an improved therapeutic index, reduced side effects, and a lower potential for drug-drug interactions. The serum concentration of NDM LMP achieved with steady-state dosing of LMP is similar to the serum concentration of the parent compound.

NDM LMP is expected to exhibit the same therapeutic properties as its parent LMP. These properties include an anti-dopaminergic, anti-serotonergic, anti-histaminic, anti-α adrenergic, and anti-muscarinic effects, as known to one skilled in the art and as described in, for example, Goodman and Gilman's The Pharmacologic Basis of Therapeutics, Eighth Edition, Pergamon Press, Elmsford, New York 1990, the relevant sections of which are incorporated by reference herein. Based on receptor ligand binding studies, these effects include but are not limited to analgesic, antiemetic, antipsychotic, sedative, anxiolytic, antisialogogic, amnesic, antihypertensive effects, migraine therapy, and control of symptoms of benign prostatic hyperplasia (BPH).

NDM LMP binds to the same receptors and with the same affinity (extent) as its parent; that is, NDM LMP has a comparable binding affinity constant for receptors as the parent compound. Administration of the metabolite NDM LMP desirably minimizes or eliminates the sulfoxide metabolite that forms when the parent LMP is administered, for example, by oral administration. The α₁ adrenergic and histaminic antagonist effects that are elicited by the sulfoxide metabolite may thus be minimized or eliminated. For a patient, administration of NDM LMP instead of the parent LMP may thus desirably minimize the drowsiness and lowered blood pressure potential due to the sulfoxide metabolite which is formed from the metabolism of LMP.

NDM LMP exhibits relatively high binding to the following receptors: dopamine, such as D₂ and D₃ receptors; serotonin, such as 5-HT_{2A} and 5-HT_{2C} receptors; α adrenergic, such as α_{1A}, α_{1B}, and α_{1C} plus α_{2A}, α_{2B}, and α_{2c} receptors; muscarinic, such as M₁, M₂, M₃, M₄, and M₅; and histamine receptors such as H₁. A pharmaceutical composition containing NDM LMP, an antagonist for these receptors, would thus be expected to affect the functions regulated by these receptors. A dopamine and serotonin receptor antagonist regulates mood and satiety. An α₁ adrenergic receptor antagonist regulates blood pressure. An α₂ adrenergic receptor antagonist regulates suppressing sympathetic output, increasing vagal tone, facilitating platelet aggregation, inhibiting the release of norepinephrine and acetylcholine from nerve endings, and metabolic effects including suppression of insulin secretion. One of the many pharmacologic actions of a histamine receptor antagonist is the prevention or relief of itching (anti-pruritic effects) when it binds to peripheral histiminic receptors, and causes sedation when it binds to central nervous system (CNS) histiminic receptors.

NDM LMP administered clinically is expected to exhibit properties which provide improved clinical effects relative to effects seen when the parent compound is administered. Specifically, the NDM LMP metabolite exhibits less biotransformation and may have a greater bioavailability than its parent pursuant to hepatic metabolic and absorption assay studies. NDM LMP thus has improved oral absorption, reduced clearance variability, an improved therapeutic index, reduced side effects and a lower potential for drug-drug interactions.

NDM LMP may be synthesized starting from 2-methoxy phenothiazine or its parent levomepromazine (EP grade), which is commercially available, for example, from Aventis (Vitry, France), Orgasynth (Paris, France) or Egis Pharmaceuticals (Budapest, Hungary). Synthesis pathways are known to one skilled in the art. Like its parent, it may be synthesized as a base or as a salt, such as a maleate or hydrochloride salt. For example, one synthetic scheme condenses (RS)-monomethylamino-2-methyl-3-chloropropane with 2-methoxy phenothiazine in toluene solution. The resultant solution is treated with sulfuric acid and sodium hydroxide for purification. The optical isomers are separated by selective crystallization with tartaric acid to obtain the dextrorotatory R enantiomer of the NDM LMP tartaric acid. The aqueous solution of NDM LMP tartrate is then reacted with maleic anhydride to obtain NDM LMP as R(+)-N-desmethyl levomepromazine maleate. R(+)-N-desmethyl levomepromazine maleate is crystallized, isolated and dried. NDM LMP may also be obtained from commercial sources, e.g., LGC Promochem (Wesel, Germany), or may be isolated as an intermediate of levomepromazine metabolism.

NDM LMP is formulated into pharmaceutically acceptable compositions for human or veterinary use; that is, a human or a non-human animal. Such methods are known to one skilled in the art, for example, as described in Pharmaceutical Preformulation and Formulation, Gibson, Ed., HIS Health Group, Englewood Col. (2001) and Remington's Pharmaceutical Sciences, 20^{th} Edition, 2001 (Mack Publishing Company, PA), the relevant sections of each of which is expressly incorporated by reference herein. NDM LMP may be administered as a free base or as a pharmaceutically acceptable salt, such as a maleate salt or other salts, as known to one skilled in the art. NDM LMP may be administered with other active agents. As only one example, a formulation containing NDM LMP may include any other analgesics known to one skilled in the art, such as non-steroidal anti-inflammatory agents, acetaminophen, opiate analgesics, etc. The compositions may be administered by any route, such as enteral, parenteral, topical, buccal, sublingual, intranasal, intra-spinal, intrathecal, ophthalmic, otic, inhalation, dermal, transdermal, subcutaneous, rectal, vaginal, etc. In one embodiment, NDM LMP is formulated for oral administration and is administered orally.

Enteral formulations may be solids, liquids, solutions, emulsions, suspensions, gels, etc. Solid formulations may be in any unit dosage form, such as capsules, tablets, gums, caplets, pills, powders, dispersible granules, cachets, or suppositories. Parenteral formulations may be administered subcutaneously, intravenously, intrathecally, or intramuscularly. NDM LMP may be in mixture or admixture with nontoxic pharmaceutically-acceptable excipients. For solid formulations, such excipients may be, for example, inert diluents, such as calcium carbonate, sodium carbonate, lactose, calcium phosphate, or sodium phosphate; granulating or disintegrating agents such as maize, starch, or alginic acid; binding agents such as starch, gelatin, or acacia; lubricating agents such as magnesium stearate or stearic acid. Hard gelatin capsules may contain NDM LMP in mixture or admixture with an inert solid such as calcium carbonate, calcium phosphate, or kaolin. Soft gelatin capsules may contain NDM LMP in mixture or admixture with an oil, such as olive oil or liquid paraffin. Suppositories may contain NDM LMP in mixture or admixture with binders and/or carriers such as polyalkylene glycols or triglycerides. For liquid formulations, excipients may be, for example, suspending agents or viscosity modifiers such as sodium carboxymethylcellulose, methylcellulose, hydroxypropyl methylcellulose, sodium alginate, polyvinyl pyrrolidine, gum tragaanth and gum acacia; dispersing or setting agents such as a naturally occurring phosphatide (lecithin); condensation products of ethylene oxide with, for example, polyoxyethylene sorbitol monooleate or polyoxyethylene sorbitan monooleate. The formulations may also contain one or more preservatives such as ethyl or n-propyl p-hydroxy benzoate, one or more coloring agents and/or flavoring agents; one or more sweetening agents such as sucrose, saccharin, or sodium cyclamate. In one embodiment, NDM LMP is formulated as either a solid or a liquid for oral dosing. The formulation may be an immediate release or a sustained release type. Sustained release formulations may be manufactured as known to one skilled in the art, and include coatings, microspheres, liposomes, capsules, etc.

In one embodiment, NDM LMP is formulated and/or administered to a specific effect. As one example, a formulation to achieve an antihistamine effect may contain a lower amount or concentration of NDM LMP than a formulation to achieve an antipsychotic effect. As another example, an NDM LMP formulation for benign prostate hypertrophy therapy or as an anti-emetic may contain a lower amount or concentration of NDM LMP than a formulation to achieve an antipsychotic effect. As another example, an NDM LMP formulation as an analgesic or for migraine therapy may contain a higher amount or concentration of NDM LMP than a formulation for control of symptoms of BPH or as an antiemetic, but a lower amount or concentration than a formulation as an antipsychotic or sedative. In one embodiment, a dose of NDM LMP administered to achieve an antiemetic effect and/or an antipruritic effect, and/or to control symptoms of BPH, is in the range of about 1 mg/day to about 50 mg/day for oral administration, with a fraction of this dose range for non-enteral administration based on the bioavailabilty (ƒ) of NDM LMP (

$$f = \frac{AUCpo}{AUCiv}$$

where AUCₚₒ=area under the concentration versus time curve with oral (po) administration, and AUCᵢᵥ=area under the concentration versus time curve with intravenous (iv) administration). In another embodiment, a dose of NDM LMP administered for an analgesic effect and/or migraine therapy effect is in the range of about 5 mg/day to about 250 mg/day for oral administration, with a fraction of this dose range for non-enteral administration. In another embodiment, a dose of NDM LMP administered for a sedative effect and/or an antipsychotic effect is greater than about 50 mg/day to about 1000 mg/day for oral administration, with a fraction of this dose range for non-enteral administration.

In one embodiment, NDM LMP is formulated and/or administered to achieve substantially the same serum concentration as when the parent LMP is administered; that is, the same pharmacologically active concentration of the combined LMP and NDM LMP. In another embodiment, NDM LMP is formulated and/or administered at a lower dose than the parent LMP. The serum concentration of clinically administered NDM LMP should be comparable to that achieved when the parent LMP is administered. Thus, the dose of NDM LMP to achieve this serum concentration should consider that the parent LMP, upon administration, has a bioavailability of about 20% and results in formation of both NDM LMP and the sulfoxide metabolite, other metabolites, as well as some unchanged parent LMP.

The dose of NDM LMP may differ according to the route of administration. In general, doses of NDM LMP in oral formulations are higher than doses of NDM LMP in non-enteral formulations (for example, parenteral formulations may contain about one-fifth, about one-tenth, about one-twentieth, etc. the dose in the oral formulation based on NDM LMP bioavailability). In one embodiment, an oral dose of NDM LMP may be in the range of about 1 mg daily to about 1000 mg daily, and an intramuscular or intravenous dose of NDM LMP may be a fraction of that dose, for example, from about 0.5 mg daily to about 400 mg daily. In another embodiment, an oral dose of NDM LMP may be in the range of about 1 mg daily to about 100 mg daily. In another embodiment, the NDM LMP dose may be in the range of about 1 mg per dose to about 50 mg per dose. In another embodiment, the NDM LMP dose for oral administration may be up to 250 mg per dose. A dose may be administered at any interval, as known to one skilled in the art, for example once daily, twice a day, etc.

Indications for NDM LMP administration may include therapeutic and/or palliative remedies for a variety of disorders, similar to indications for LMP administration. Thus, NDM LMP may be used to treat, relieve, reduce the severity of, reduce the occurrence of, etc. disorders which may range in severity and include, but are not limited to, psychoses, agitation, pain, migraine headache, nausea, vomiting, itching, hypertension, control of symptoms of BHP, excess gastrointestinal (GI) secretions, and sleeplessness. NDM LMP may thus have properties as an antipsychotic to treat psychoses, anxiolytic to treat anxiety, analgesic to treat pain and migraine headaches, antiemetic to treat nausea and/or vomiting, sedation to treat agitation and sleeplessness, antipruritic to treat itching, antihypertensive to treat high blood pressure, antisialogogic to dry excess gastrointestinal and respiratory secretions such as during presurgical preparation, and to control the symptoms of BPH. In addition, administration of NDM LMP may be for uses presently unknown, for example, uses which are effected by antagonist binding to as yet uncharacterized receptors. While not intending to be bound to a specific theory as to its mechanism of action, NDM LMP resembles the agents classified as atypical antipsychotic agents, in that its dopamine activity is balanced with its serotonin activity. That is, the atypical antipsychotic agents have a high affinity for many serotonergic receptors subtypes (e.g., 5-HT_{2A} and 5-HT_{2C}) which have been proposed as necessary for their effectiveness and uniqueness. In this respect NDM LMP resembles risperidone, olanzapine, quetiapine, and ziprasidone.

Its analgesic effect appears to be mediated through the central nervous system (CNS) and is not due to an opioid receptor interaction. Thus, analgesic treatment with NDM LMP occurs without the addictive potential seen with the opioid analgesics. The parenteral analgesic potency of NDM LMP is expected to be comparable to its LMP parent while its oral analgesic potency is expected to be greater than the parent.

As described, any other active agents may be included in the formulation with NDM LMP. The active agent may produce similar or different pharmacologic effects as NDM LMP. For example, in one embodiment, NDM LMP is orally administered as an analgesic at a dose of between about 5 mg/day to about 250 mg/day, or by a non-enteral route at a dose of between about 2 mg/day to about 100 mg/day, in combination with another analgesic and/or with another antiemetic. This formulation may be used to relieve both pain and the nausea that sometimes accompanies pain, for example, in treating migraine headaches.

The inventive methods and compositions will be further appreciated in view of the following examples.

Receptor-ligand binding studies were performed as known to one skilled in the art. Compounds evaluated were LMP, the parent compound, the NDM LMP metabolite of LMP, and the sulfoxide metabolite of LMP. These studies verified receptor binding and affinity by the parent compound, as well as identified other potential receptors for the parent compound and its metabolites, and identified and verified receptors and receptor affinity for the metabolites. These data permitted elucidation of the therapeutic profile for NDM LMP, that is, its efficacy and mechanism of action. These data also permitted identification of potential side effects of NDM LMP, for example, as used to generate safety information.

### EXAMPLE

Receptor binding studies were performed to determine binding affinity of the levomepromazine parent compound (LMP), the N-desmethyl levomepromazine metabolite (NDM LMP), and the sulfoxide metabolite (sulfoxide) against various receptors. Dopamine receptor affinity included binding to D₁, D₂, D₃, D₄, and D₅ receptors. Serotonin receptor affinity included binding to 5-HT_{1A}, 5-HT_{2A}, 5-HT_{2B}, 5-HT₂, 5-HT_{5A}, 5-HT₆, and 5-HT₇ receptors. Alpha adrenergic receptor affinity included binding to α_{1A}, α_{1B}, α_{1C}, α_{2A}, α_{2B}, and α_{2C} receptors. Muscarinic receptor affinity included binding to M₁, M₂, M₃, M₄, and M₅ receptors. Binding to the histamine H₁ receptor, the calcium ion channel receptor, and the sodium ion channel receptor were also evaluated. Data for each of these are shown in the following tables. In each of the tables, the following abbreviations are used: LMP indicates the parent compound levomepromazine. NDM LMP indicates the N-desmethyl (N-desmethyl) metabolite. Sulfoxide indicates the sulfoxide metabolite. Potential activity indicates that the listed activity is only a partial representation, and is a likely but not all inclusive activity. Kᵢ (the inhibition constants of binding) indicate binding affinity. As known to one skilled in the art, a lower value for Kᵢ indicates less inhibition of binding, and hence greater binding. − indicates no detectable activity.

TABLE 1
DOPAMINE RECEPTOR AFFINITY

| | LMP | NDM LMP | Sulfoxide | |
|---|---|---|---|---|
| | Ki | Ki | Ki | Potential |
| Receptors | (nM) | (nM) | (nM) | Activity |
| Dopamine D₁ | 36 | 99 | — | sympatholytic |
| Dopamine D₂ | 8 | 17 | — | neuroleptic & |
| | | | | extrapyramidal |
| | | | | symptoms |
| Dopamine D₃ | 4 | 5 | — | |
| Dopamine D_{4.2} | 769 | 3110 | — | behavioral/CNS |
| Dopamine D₅ | 180 | 243 | — | sympatholytic |

TABLE 2
SEROTONIN RECEPTOR AFFINITY

| | LMP | NDM LMP | Sulfoxide | |
|---|---|---|---|---|
| | Ki | Ki | Ki | Potential |
| Receptors | (nM) | (nM) | (nM) | Activity |
| Serotonin 5-HT_{1A} | 827 | 641 | — | behavioral reactivity |
| Serotonin 5-HT_{2A} | <4 | <4 | 410 | dopamine release |
| | | | | NS* |
| Serotonin 5-HT_{2B} | 19 | 27 | — | anxiolytic |
| Serotonin 5-HT_{2C} | 8 | 9 | 1980 | 2A-like; hunger |
| Serotonin 5-HT_{5A} | 136 | 152 | — | behavioral reactivity |
| Serotonin 5-HT₆ | 82 | 98 | — | cognition enhanced |
| Serotonin 5-HT₇ | 24 | 18 | — | vascular/GI + tone |

*NS = nigra substantia

TABLE 3
ADRENERGIC RECEPTOR AFFINITY

| | LMP | NDM LMP | Sulfoxide | |
|---|---|---|---|---|
| | Ki | Ki | Ki | Potential |
| Receptors | (nM) | (nM) | (nM) | Activity |
| Adrenergic α_{1A} | 2 | 2 | 141 | orthostatic BP |
| Adrenergic α_{1B} | 2 | 2 | 80 | orthostatic BP |
| Adrenergic α_{1C} | 2 | 2 | 193 | |
| Adrenergic α_{2A} | 99 | 239 | — | |
| Adrenergic α_{2B} | 25 | 48 | — | |
| Adrenergic α_{2C} | 77 | 74 | — | |

TABLE 4
MUSCARINIC RECEPTOR AFFINITY

| | LMP | NDM LMP | Sulfoxide | |
|---|---|---|---|---|
| | Ki | Ki | Ki | Potential |
| Receptors | (nM) | (nM) | (nM) | Activity |
| Muscarinic M₁ | 43 | 54 | 726 | BP & GI secretion |
| Muscarinic M₂ | 263 | 325 | 1290 | tachycardia |
| Muscarinic M₃ | 39 | 47 | 551 | constipation |
| Muscarinic M₄ | 34 | 88 | 318 | CNS D₁ stimulation |
| Muscarinic M₅ | 61 | 60 | 481 | D₁ stimulation |

TABLE 5
HISTAMINE & ION CHANNEL RECEPTOR
AFFINITY

| | LMP | NDM LMP | Sulfoxide | |
|---|---|---|---|---|
| | Ki | Ki | Ki | Potential |
| Receptors | (nM) | (nM) | (nM) | Activity |
| Histamine H₁ | 2 | 2.1 | 6.1 | sedation, antipruritis |
| Calcium Channel | 407 | 49 | 9090 | hemodynamic and |
| | | | | cardiac conduction |
| Sodium Channel | 1090 | 643 | — | |

Metabolism of the parent LMP to the NDM LMP and sulfoxide metabolites has been evaluated. When a dose of 25 mg LMP was administered intramuscularly, no sulfoxide was detected. In a bioavailability study, when LMP was administered intravenously, sulfoxide formation was detected although to a much lower extent compared to the oral formulation. These results were the same as previously described by Dahl, Clin. Pharm. Therapeutics 19:435 (1976), suggesting very little systemic metabolism of LPM to sulfoxide. In contrast, when a dose of 50 mg of LMP was administered orally, the sulfoxide metabolite was detected at serum concentrations which were 1.5 fold to 3 fold greater than LMP concentrations. In another study, LMP in an amount ranging from 50 mg to 350 mg was orally administered on a daily basis for one week. The concentrations of the sulfoxide metabolite detected were 2.5 times greater than the concentrations of the parent compound in a recent absolute bioavailability study. The parent LMP demonstrated an absolute bioavailability of approximately 20%, when comparing a 25 mg oral formulation to a 25 mg intravenous dose. A syrup formulation of LMP produced sulfoxide concentrations greater than that observed with the tablet formulation.

The in vitro metabolism of NDM LMP is shown in FIG. 3. Primary human hepatocytes were used to evaluate the Phase I and Phase II potential biotransformation of NDM LMP. NDM LMP is metabolized to only one putative primary amine metabolite, namely N-didesmethyl levomepromazine. In addition, NDM LMP did not appear to undergo any Phase II biotransformation (i.e. conjugation), and no detectable sulfoxide metabolites were formed. NDM LMP thus was much more metabolically stable than its LMP parent compound.

In one embodiment, the oral absorption characteristics of NDM LMP are superior to the parent LMP. An absorption assay for intestinal absorption using a human colon carcinoma cell line (CACO-2 Model) has demonstrated that the apical to basolateral permeability coefficient for NDM LMP is 1.5 fold greater than the parent LMP with less variability over the dosage range. NDM LMP is influenced and transported by P-glycoprotein (p-gp) to a lesser degree than the parent LMP. The relative efflux to influx ratio of NDM LMP through the enterocyte is at least 50% less than the parent LMP. In the presence of a p-gp inhibitor (verapamil) the inhibitor of efflux for NDM LMP is at least one-half lower than the parent LMP. Gut wall (enterocyte) metabolism occurs to a lower extent with NDM LMP compared to the parent LMP. The percent of NDM LMP recovered through the system is 1.5-2.0 fold greater than that observed with the parent LMP.

With respect to the binding affinity to dopamine receptors by LMP, NDM LMP, and sulfoxide, LMP and NDM LMP demonstrated very high affinity to both D₂ and D₃ receptors. As shown in Table 1, the parent LMP had a binding affinity to D₂ of 8 nM, and to D₃ of 4 nM. The NDM LMP metabolite had a binding affinity to D₂ of 17 nM, and to D₃ of 5 nM. The sulfoxide metabolite had essentially no binding affinity to dopamine receptors.

With respect to the binding affinity to serotonin receptors by LMP, NDM LMP, and sulfoxide, LMP and NDM LMP demonstrated very high affinity to both 5-HT_{2A} and 5-HT_{2C} receptors. As shown in Table 2, the parent LMP had a binding affinity to 5-HT_{2A} of less than 4 nM, and a binding affinity to 5-HT_{2C} of 8 nM. The NDM LMP metabolite had a binding affinity to 5-HT_{2A} of less than 4 nM, and to 5-HT_{2C} of 9 nM. The sulfoxide metabolite had essentially no binding affinity to serotonin receptors.

With respect to the binding affinity to adrenergic receptors by LMP, NDM LMP, and sulfoxide, each exhibited high binding to all α₁ adrenergic receptors (α_{1A}, α_{1B}, and α_{1C} receptors), with LMP and NDM LMP exhibiting the most binding. For binding affinity to a2 adrenergic receptors, only LMP and NDM LMP demonstrated binding; no affinity was detected for binding of the sulfoxide to the α₂ receptors. The binding to α₂ adrenergic receptors by LMP and NDM LMP was comparable, but the binding of each to α₂ receptors was less than its binding to α₁ receptors.

With respect to the binding affinity to muscarinic receptors by LMP, NDM LMP, and sulfoxide, LMP and NDM LMP demonstrated moderate-to-high affinities to all receptors. Sulfoxide demonstrated low affinity to all receptors.

With respect to the binding affinity to the histamine H₁ receptor, and calcium ion channel and potassium ion channel receptors, by LMP, NDM LMP, and sulfoxide, LMP and the metabolites (NDM LMP and sulfoxide) all exhibited high affinity to H₁ receptors (Kᵢ for NDM LMP=2.1 nM; Kᵢ for sulfoxide=6.1 nM). NDM LMP had a greater affinity for both calcium channel receptors and sodium channel receptors than LMP (for calcium ion channel receptors, Kᵢ for NDM LMP=49 nM; Kᵢ for LMP=407 nM; for sodium ion channel receptors, Kᵢ for NDM LMP=643 nM; Kᵢ for LMP=1090 nM). The sulfoxide metabolite had substantially no affinity for calcium ion channel receptors (Kᵢ=9090), and no affinity was detected for sodium ion channel receptors.

The overall results of the receptor-ligand affinity and binding for LMP and the NDM LMP and sulfoxide metabolites are summarized in the following table where ≧ indicates greater or equal affinity, = indicates about the same affinity, and − indicates no affinity.

TABLE 6
LEVOMEPROMAZINE & METABOLITES
RECEPTOR AFFINITY SUMMARY

| Receptor | Relative Affinity |
|---|---|
| Dopamine | LMP = NDM LMP; − sulfoxide |
| Serotonin | LMP = NDM LMP; − sulfoxide |
| α₁-Adrenergic | LMP = NDM LMP ≧ sulfoxide |
| α₂-Adrenergic | LMP = NDM LMP; − sulfoxide |
| Histamine | LMP = NDM LMP = sulfoxide |
| Muscarinic | LMP = NDM LMP > sulfoxide |
| Calcium channel | NDM LMP > LMP > sulfoxide |
| Sodium channel | NDM LMP > LMP; − sulfoxide |

The affinity of the parent LMP and the NDM LMP metabolite was about equal for each of the following receptors: dopamine, serotonin, α adrenergic, histamine, and muscarinic receptors. The affinity of the NDM LMP metabolite exceeded the affinity of the parent compound for sodium ion and calcium ion channel receptors.

The sulfoxide metabolite exhibited no receptor affinity, relative to the LMP parent compound and the NDM LMP metabolite, for dopamine, serotonin, α₂ adrenergic, sodium channel, and calcium channel receptors. The sulfoxide metabolite exhibited decreased receptor affinity, relative to the parent compound and the NDM LMP metabolite, for α₁ adrenergic and muscarinic receptors. The sulfoxide metabolite exhibited about the same receptor affinity, relative to the parent compound and the NDM LMP metabolite, for histamine H₁ receptors. Significant clinical histamine and α₁ receptor antagonism is expected from LMP sulfoxide; LMP sulfoxide exhibits a 20 fold greater free fraction (less protein binding) and it achieves total serum concentrations that are 1.5-3.0 fold greater than either the parent LMP or NDM LMP.

These data indicate the usefulness of clinical formulations of NDM LMP for administering to a patient. For example, specific effects of NDM LMP activity may be targeted, for example, in a dose-specific manner. Also, the lowering of blood pressure (orthostatic hypotension) and drowsiness (sedation) produced by the sulfoxide metabolite may be reduced. This may be because the sulfoxide metabolite is not formed; there is no LMP parent compound present to be metabolized to the sulfoxide. Thus, these potentially undesirable effects may be minimized. Other variations or embodiments of the invention will also be apparent to one of ordinary skill in the art from the above figures, description, and examples. Thus, the forgoing embodiments are not to be construed as limiting the scope of this invention.

1. A therapeutic method comprising administering to a patient N-desmethyl levomepromazine (NDM LMP) in a pharmaceutically acceptable formulation for providing at least one of a dopaminic antagonist effect, a serotonergic antagonist effect, an α adrenergic antagonist effect, a histaminic antagonist effect, a muscarinic antagonist effect, a sodium ion channel antagonist effect, or a calcium ion channel antagonist effect.

2. The method of claim 1 wherein NDM LMP is administered to achieve at least one of an antiemetic effect, an antiprutic effect, or to control symptoms of BPH at a dose in the range of about 1 mg/day to about 50 mg/day for enteral administration or at a fraction thereof for non-enteral administration as a function of the absolute bioavailability value of NDM LMP.

3. The method of claim 1 wherein NDM LMP is administered to achieve at least one of an analgesic effect or migraine therapy effect at a dose in the range of about 5 mg/day to about 250 mg/day for enteral administration or at a fraction thereof for non-enteral administration as a function of the absolute bioavailability value of NDM LMP.

4. The method of claim 1 wherein NDM LMP is administered to achieve at least one of an antipsychotic effect, a sedative effect, or an anxiolytic effect at a dose in the range of about 50 mg/day to about 1000 mg/day for enteral administration or at a fraction thereof for non-enteral administration as a function of the absolute bioavailability value of NDM LMP.

5. The method of claim 1 administering NDM LMP at a dose effective to achieve substantially the same steady state serum concentration as achieved by LMP as combined LMP and NDM LMP serum concentration when LMP is administered by the same route.

6. The method of claim 1 wherein NDM LMP is administered at a relatively lower dose as an antiemetic, antipruritic, and to control symptoms of BPH, at a relatively higher dose as an antipsychotic, sedative, and anxiolytic, and at a dose higher than the lower dose and lower than the higher dose as an analgesic.

7. The method of claim 1 wherein NDM LMP is administered in an amount ranging between about 5 mg to about 250 mg for an antihypertensive effect.

8. A therapeutic method comprising providing to a patient a composition producing substantially the same pharmaceutical effects as levomepromazine (LMP) by administering to the patient N-desmethyl levomepromazine (NDM LMP) in a pharmaceutically acceptable formulation.

9. The method of claim 8 wherein NDM LMP is administered at a dose effective to achieve substantially the same steady state serum concentrations as achieved by LMP as combined LMP and NDM LMP serum concentration when LMP is administered by the same route.

10. The method of claim 8 wherein the pharmaceutical effects of a sulfoxide LMP metabolite are reduced.

11. A therapeutic method comprising administering to a patient a composition comprising N-desmethyl levomepromazine (NDM LMP) in a pharmaceutically acceptable formulation for providing a dopaminic antagonist effect.

12. The method of claim 11 wherein the antagonist effect is to at least one of D₁ receptors, D₂ receptors, D₃ receptors, or D₅ receptors.

13. A therapeutic method comprising administering to a patient a composition comprising N-desmethyl levomepromazine (NDM LMP) in a pharmaceutically acceptable formulation for providing a serotonergic antagonist effect.

14. The method of claim 13 wherein the antagonist effect is to at least one of 5-HT_{2A} receptors, 5HT_{2C} receptors, 5-HT_{2B1} receptors, 5-HT_{5A} receptors, or 5-HT₇ receptors.

15. A therapeutic method comprising administering to a patient a composition comprising N-desmethyl levomepromazine (NDM LMP) in a pharmaceutically acceptable formulation for providing a α adrenergic antagonist effect.

16. The method of claim 15 wherein the antagonist effect is to at least one of α_{1A} receptors, α_{1B} receptors, α_{1C} receptors, α_{2A} receptors, α_{2B} receptors, or α_{2C} receptors.

17. A therapeutic method comprising administering to a patient a composition comprising N-desmethyl levomepromazine (NDM LMP) in a pharmaceutically acceptable formulation for providing a histiminic antagonist effect.

18. The method of claim 17 wherein the antagonist effect is to H₁ receptors.

19. A therapeutic method comprising administering to a patient a composition comprising N-desmethyl levomepromazine (NDM LMP) in a pharmaceutically acceptable formulation for providing a muscarinic antagonist effect.

20. The method of claim 19 wherein the antagonist effect is to at least one of M₁ receptors, M₂ receptors, M₃ receptors, M₄ receptors, or M₅ receptors.

21. A therapeutic method comprising administering to a patient a composition comprising N-desmethyl levomepromazine (NDM LMP) in a pharmaceutically acceptable formulation for providing an ion channel antagonist effect.

22. The method of claim 21 wherein the ion channel is a sodium ion channel, a calcium ion channel, or both a sodium ion channel and a calcium ion channel.

23. A therapeutic method comprising orally administering to a patient N-desmethyl levomepromazine (NDM LMP) in a pharmaceutically acceptable formulation for therapy with reduced α adrenergic antagonist effects and reduced histaminic antagonist effects relative to administering levomepromazine.

24. The method of claim 23 wherein the reduced effects include at least one of sedation or hypotension.

25. A therapeutic method comprising administering to a patient a pharmaceutical composition comprising N-desmethyl levomepromazine (NDM LMP) in an effective amount for levomepromazine therapy substantially free of a sulfoxide metabolite of levomepromazine.

26. A composition comprising an amount of an isolated N-desmethyl levomepromazine (NDM LMP) in a pharmaceutically acceptable formulation to provide at least one of an analgesic, antiemetic, antipsychotic, sedative, anxiolytic, antisialogogic, amnesic, antihypertensive, anti-pruritic, migraine therapy, or control of symptomatic benign prostatic hyperplasia effect.

27. The composition of claim 26 containing NDM LMP at a dose in the range of about 1 mg/day to about 1000 mg/day formulated for oral administration.

28. The composition of claim 26 containing NDM LMP at a dose in the range of about 0.5 mg/day to about 400 mg/day formulated for parenteral administration.

29. The composition of claim 26 containing NDM LMP at a dose in the range of about 5 mg/day to about 250 mg/day formulated for oral administration.

30. The composition of claim 26 containing NDM LMP at a dose in the range of about 1 mg/day to about 50 mg/day formulated for oral administration.

31. The composition of claim 26 containing NDM LMP at a dose in the range of about 50 mg/day to about 1000 mg/day formulated for oral administration.

32. The composition of claim 26 containing NDM LMP at a dose up to about 250 mg/day for oral administration.

33. The composition of claim 26 formulated for at least one of human use or veterinary use.

34. The composition of claim 26 in a formulation chosen from at least one of oral, injectable, topical, dermal, transdermal, buccal, sublingual, intranasal, intraspinal, intrathecal, ophthalmic, otic, inhalation, rectal, or vaginal.

35. The composition of claim 26 wherein the formulation is chosen from at least one of a solid, a liquid, a solution, an emulsion, a suspension, a syrup, an elixir, a gel, a capsule, a tablet, a gum, a caplet, a pill, a powder, a granule, or a cachet.

36. The composition of claim 26 substantially free of a sulfoxide levomepromazine metabolite.

37. A composition comprising isolated N-desmethyl levomepromazine (NDM LMP) in a pharmaceutically acceptable formulation at a dose in the range of about 1 mg/day to about 1000 mg/day formulated for enteral administration as a function of the absolute bioavailability value of NDM LMP for non-enteral formulation.
